(19) United States
(12) Patent Application Publication
(10) Pub. No.: US 2013/0184513 A1
(43) Pub. Date: Jul. 18, 2013

(54) SKIN CARE METHOD AND SKIN CARE DEVICE

(71) Applicants: Emi Saito, Osaka-shi (JP); Misaki Nakamura, Osaka-shi (JP)

(72) Inventors: Emi Saito, Osaka-shi (JP); Misaki Nakamura, Osaka-shi (JP)

(73) Assignee: SHARP KABUSHIKI KAISHA, Osaka-shi, Osaka (JP)

(21) Appl. No.: 13/824,779

(22) PCT Filed: Sep. 13, 2011
(86) PCT No.: PCT/JP2011/070802

§ 371 Date: Mar. 18, 2013

(30) Foreign Application Priority Data
Nov. 30, 2010 (JP) 2010-266622

(51) Int. Cl. A61N 5/10

(52) U.S. Cl. CPC A61N 5/10; A61N 2005/1087
USPC 600/1

(57) ABSTRACT

A skin care method using a closed-bottomed casing 2 having an open surface 2a and an ion generating unit 10 in which discharge is generated in the air by applying a high voltage to generate positive ions and negative ions in the casing 2 includes driving the ion generating unit 10 while the open surface 2a is closed by a skin surface to seal the inside of the casing 2, and bringing ions generated in the ion generating unit 10 into contact with the skin surface.

### TECHNICAL FIELD

The present invention relates to a skin care method and a skin care device that suppress an increase in sebum of the skin.

### BACKGROUND ART

Recently, people's awareness of beauty has been increasing, and, regardless of men and women, the needs for beautiful skin in which pores of the human face are not conspicuous have been increasing. Sebum on the skin surface enters pores, and peroxides produced by oxidation of the sebum accumulate in the pores. Consequently, the pores are opened and become conspicuous.

PTL 1 discloses a skin care method, the method including applying an adhesive sheet onto the face, and physically peeling off peroxides that have accumulated in pores of the skin. PTL 2 discloses a skin care method, the method including applying a cleansing pack onto the face so as to allow peroxides in pores of the skin to adsorb to the cleansing pack, and washing the peroxides away.

### CITATION LIST

### Patent Literature

PTL 1: Japanese Unexamined Patent Application Publication No. 7-330575 (pp. 3-7, FIG. 3)
PTL 2: Japanese Unexamined Patent Application Publication No. 7-173033 (pp. 2-3)
PTL 3: Japanese Unexamined Patent Application Publication No. 9-215724 (pp. 3-5, FIG. 1)

### SUMMARY OF INVENTION

### Technical Problem

However, according to the skin care method disclosed in PTL 1, the keratin layer, which is an indispensable layer of the skin, is also peeled off because the sheet closely adheres to the skin. This causes a problem in that a large stress is applied to the skin. According to the skin care method disclosed in PTL 2, the cleansing pack is washed away after the application thereof, thus taking time and causing problems in terms of convenience.

An object of the present invention is to provide a skin care method and a skin care device, the method and the device being capable of suppressing the conspicuousness of pores while the stress on the skin is low and an increase in sebum is easily suppressed.

### Solution to Problem

In order to achieve the above object, the skin care method of the present invention using a closed-bottomed casing having an open surface and an ion generating unit in which discharge is generated by applying a high voltage to generate positive ions and negative ions in the casing includes driving the ion generating unit while the open surface is closed by a skin surface to seal the inside of the casing, and bringing ions generated in the ion generating unit into contact with the skin surface.

With this structure, the open surface of the casing is closed by a skin surface, and the casing is filled with the positive ions and the negative ions generated in the ion generating unit. The positive ions and the negative ions contact the skin surface, and active species generated by the ions also contact the skin surface. Accordingly, an increase in sebum on the skin surface is suppressed and Staphylococcus aureus on the skin surface is decomposed.

In the skin care method according to the present invention, the positive ions may be represented by H⁺(H₂O)ₘ (where m is any natural number) and the negative ions may be represented by O₂⁻(H₂O)ₙ (where n is any natural number). With this structure, H⁺(H₂O)ₘ and O₂⁻(H₂O)ₙ generated in the ion generating unit chemically react with each other to generate active species of hydrogen peroxide (H₂O₂) and a hydroxyl radical (.OH), which have a high oxidizing power.

In the skin care method according to the present invention, the ions may be allowed to naturally diffuse in the casing.

In the skin care method according to the present invention, the ions may be generated while the skin surface is warmed. With this structure, pores are opened by warming the skin surface to suppress an increase in sebum in the pores.

A skin care device according to the present invention includes a closed-bottomed casing having an open surface, and an ion generating unit in which discharge is generated by applying a high voltage to generate positive ions and negative ions in the casing, wherein ions are generated from the ion generating unit which is driven while the inside of the casing is sealed by closing the open surface with a skin surface to bring the ions into contact with the skin surface.

With this structure, the open surface of the casing is closed by the skin surface, and the casing is filled with the positive ions and the negative ions generated in the ion generating unit. The positive ions and the negative ions contact the skin surface, and active species generated by the ions also contact the skin surface. Accordingly, an increase in sebum on the skin surface is suppressed and Staphylococcus aureus on the skin surface is decomposed.

In the skin care device according to the present invention, the positive ions may be represented by H⁺(H₂O)ₘ (where m is any natural number) and the negative ions may be represented by O₂⁻(H₂O)ₙ (where n is any natural number).

In the skin care device according to the present invention, the ions may be allowed to naturally diffuse in the casing. With this structure, blowing with a fan or the like is not conducted in the casing and thus drying of the skin surface and the eyes can be prevented.

The skin care device according to the present invention may further include a warming device that warms the skin surface that contacts the ions. With this structure, the skin surface is warmed by the warming device to open pores, and an increase in sebum in the pores is suppressed.

In the skin care device according to the present invention, the casing may have a shape of a mask that covers a human face, and the warming device may be arranged along a forehead portion and a nose portion of the face. With this structure, pores in the forehead and nose portions are opened to suppress an increase in sebum in the pores.

In the skin care device according to the present invention, the casing may have a cup shape, and a peripheral edge of the open surface may be pressed onto the skin surface by holding the casing. With this structure, the peripheral edge of the open surface is pressed onto the skin surface such as the face by holding the cup-shaped casing.

In the skin care device according to the present invention, the casing may have a shape of a mask that covers a human face, and packing may be provided around a peripheral edge of the open surface. With this structure, the packing is fitted to the face to close the open surface of the mask-shaped casing.

### Advantageous Effects of Invention

According to the present invention, since positive ions and negative ions are generated and brought into contact with a skin surface while an open surface of a casing is closed by the skin surface, the conspicuousness of pores can be suppressed while the stress on the skin is low and an increase in sebum is easily suppressed. Furthermore, active species generated by the ions decompose Staphylococcus aureus on the skin surface, thus preventing skin roughening.

### BRIEF DESCRIPTION OF DRAWINGS

FIG. 1 is a perspective view illustrating a skin care device according to a first embodiment of the present invention.

FIG. 2 is a front view illustrating the skin care device according to the first embodiment of the present invention.

FIG. 3 is a side view illustrating the skin care device according to the first embodiment of the present invention.

FIG. 4 is a perspective view illustrating an ion generating unit of the skin care device according to the first embodiment of the present invention.

FIG. 5 is a side view illustrating a relevant part of the ion generating unit of the skin care device according to the first embodiment of the present invention.

FIG. 6 is a perspective view illustrating another ion generating unit of the skin care device according to the first embodiment of the present invention.

FIG. 7 is a graph explaining an effect of suppressing an increase in sebum by ions.

FIG. 8 is a graph explaining an effect of suppressing the proliferation of Staphylococcus aureus by ions.

FIG. 9 is a flowchart illustrating an operation of the skin care device according to the first embodiment of the present invention.

FIG. 10 is a perspective view illustrating a skin care device according to a second embodiment of the present invention.

FIG. 11 is a back view illustrating the skin care device according to the second embodiment of the present invention.

FIG. 12 is a side view illustrating a state in which the skin care device according to the second embodiment of the present invention is worn.

### DESCRIPTION OF EMBODIMENTS

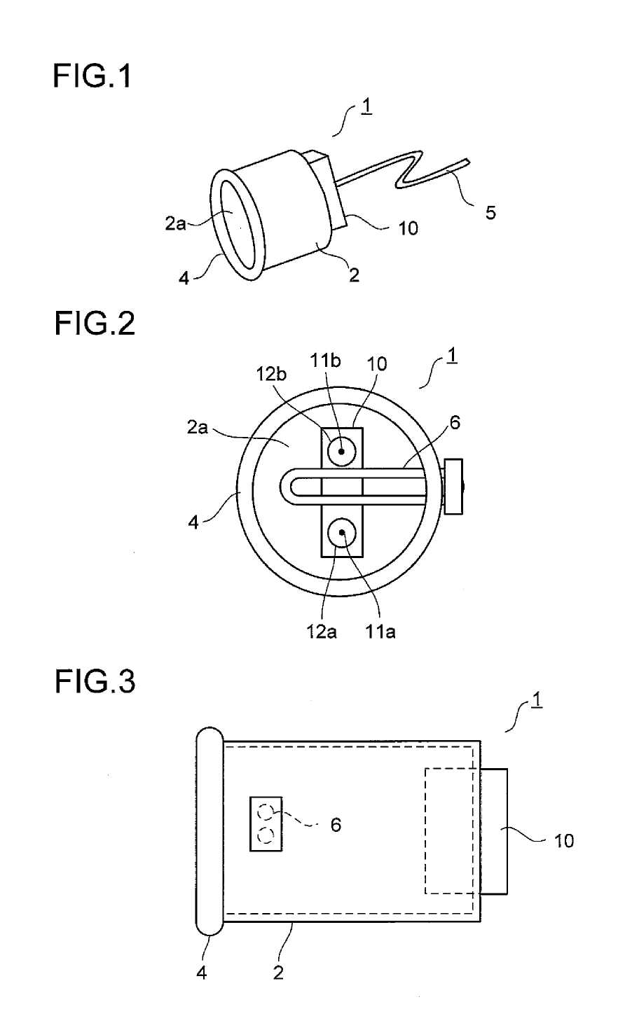

Embodiments of the present invention will now be described with reference to the drawings. FIGS. 1, 2, and 3 are respectively a perspective view, a front view, and a side view that illustrate a skin care device according to a first embodiment. A skin care device 1 includes a casing 2 formed so as to have a cylindrical, closed-bottomed cup shape having an open surface 2a on one surface thereof. Packing 4 composed of an elastomer such as silicone rubber is provided around a peripheral edge of the open surface 2a. The packing 4 is closely attached to a skin surface to close the open surface 2a, thus sealing the inside of the casing 2.

A heater 6 and an ion generating unit 10 to which electric power is supplied through a power cord 5 are attached to the casing 2. The heater 6 is formed of a glass tube heater or the like arranged in the casing 2, and generates heat of up to about 50° C. so as to prevent scalding. The heater 6 constitutes a warming device that warms the skin surface closing the open surface 2a.

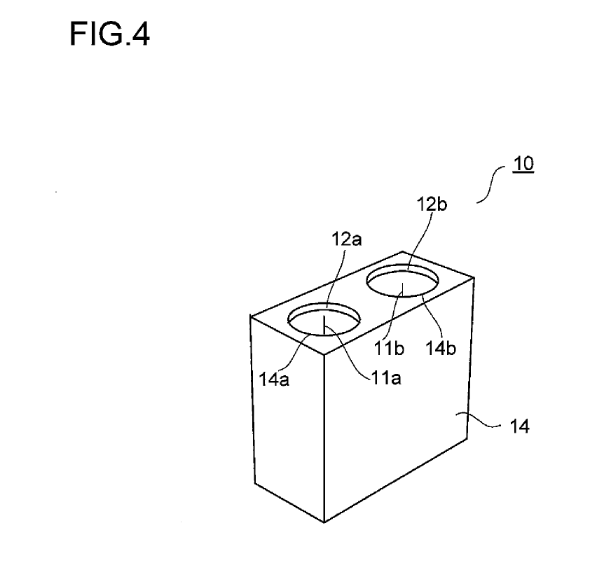

FIG. 4 is a perspective view of the ion generating unit 10. The ion generating unit 10 is covered with a body case 14. Ion release holes 14a and 14b each having a diameter of, for example, about 8 mm are opened in the top surface of the body case 14. Needle-shaped discharge electrodes 11a and 11b are arranged in the release holes 14a and 14b, respectively. Ring-shaped counter electrodes 12a and 12b that are respectively arranged around the peripheries of the discharge electrodes 11a and 11b so as to face the discharge electrodes 11a and 11b are arranged along inner surfaces of the release holes 14a and 14b, respectively.

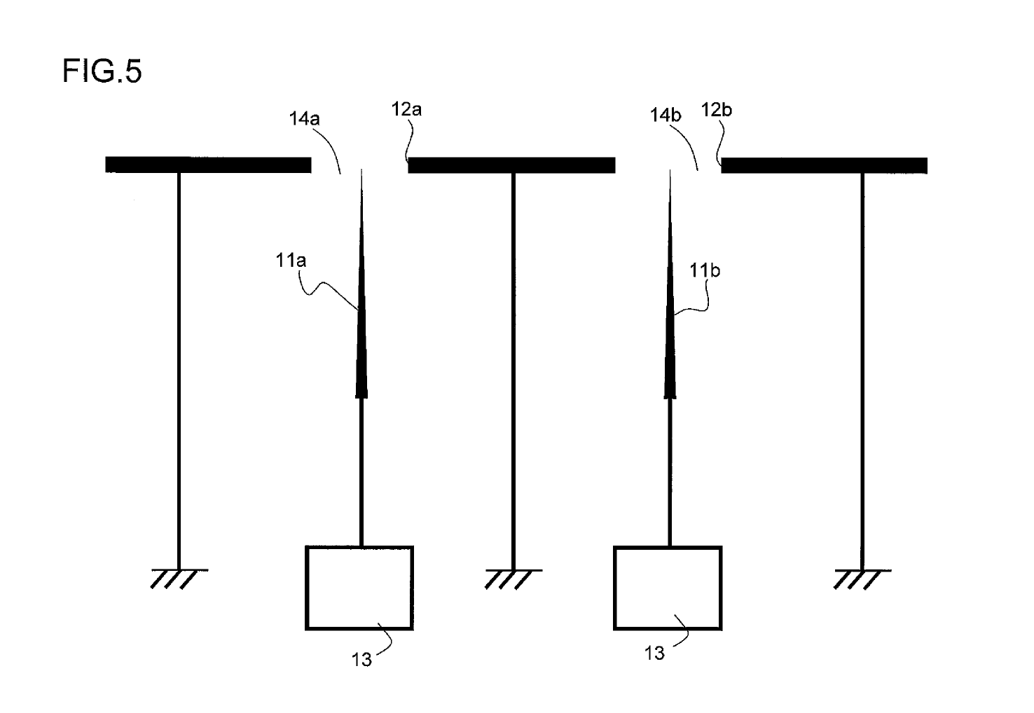

FIG. 5 is a side view illustrating the details of electrode portions of the ion generating unit 10. The counter electrodes 12a and 12b are grounded and the discharge electrodes 11a and 11b are connected to high-voltage power supplies 13 arranged in the body case 14.

One of the high-voltage power supplies 13 connected to the discharge electrode 11a generates a positive high pulse voltage (for example, at a frequency of 60 Hz and a peak voltage of about 2 kV). The other high-voltage power supply 13 connected to the discharge electrode 11b generates a negative high pulse voltage (for example, at a frequency of 60 Hz and a peak voltage of about 2 kV). With this structure, electric discharge occurs between a tip of the discharge electrode 11a and the counter electrode 12a and between a tip of the discharge electrode 11b and the counter electrode 12b, thus generating plasma. Molecules of oxygen (O₂), water (H₂O), etc. in the air receive energy from the generated plasma.

In the case where the applied voltage is a positive voltage, water molecules in the air are ionized to generate hydrogen ions (H⁺). The hydrogen ions form clusters with water molecules in the air by solvation energy, and positive ions represented by H⁺(H₂O)ₘ (where m is any natural number) are mainly generated. The positive ions are released from the release hole 14a in which the discharge electrode 11a is arranged.

In the case where the applied voltage is a negative voltage, oxygen molecules or water molecules in the air are ionized to generate oxygen ions O₂⁻. The oxygen ions form clusters with water molecules in the air by solvation energy, and negative ions represented by O₂⁻(H₂O)ₙ (where n is any natural number) are mainly generated. The negative ions are released from the release hole 14b in which the discharge electrode 11b is arranged.

In this case, it is necessary to provide energy of at least 5.12 eV, which is the formation energy of a hydrogen ion (H⁺), by applying a positive voltage. It is necessary to provide energy of at least 0.44 eV, which is the formation energy of an oxygen ion (O₂⁻), by applying a negative voltage. On the other hand, the formation energy of nitrogen oxide ions (such as NO₂⁻ and NO₃⁻), which are generated from nitrogen in the air and harmful to the human body, is 9.76 eV. Therefore, it is necessary to apply a voltage lower than the voltage that provides an energy of 9.76 eV. The formation energy of ozone (O₃) is 5.12 eV. With an increase in the energy, the amount of ozone generated also increases.

Accordingly, the positive voltage applied to the discharge electrode 11a is set to a voltage that provides energy of 5.12 eV or more and close to 5.12 eV. The negative voltage applied to the discharge electrode 11b can be set to a voltage that provides energy of 5.12 eV or more and close to 5.12 eV as in the case of the positive voltage. The negative voltage is more preferably set to a voltage that provides energy of 0.44 eV or more and less than 5.12 eV. In this case, H⁺(H₂O)ₘ and O₂⁻(H₂O)ₘ can be mainly generated while suppressing the generation of nitrogen oxides and ozone harmless to the human body.

As shown in formulae (1) to (3), H⁺(H₂O)ₘ and O₂⁻(H₂O)ₙ generate a hydroxyl radical (—OH) and hydrogen peroxide (H₂O₂), which are active species having a high oxidizing power, in the air by collision. Herein, m′ and n′ are each any natural number. The active species such as a hydroxyl radical and hydrogen peroxide decompose Staphylococcus aureus.

H⁺(H₂O)ₘ+O₂⁻(H₂O)ₙ→.OH+1/2O₂+(m+n)H₂O (1)

H⁺(H₂O)ₘ+H⁺(H₂O)_{m′}+O₂⁻(H₂O)ₙ+O₂⁻(H₂O)_{n′}→2.OH+O₂+(m+m′+n+n′)H₂O (2)

H⁺(H₂O)+H⁺(H₂O)_{m′}+O₂⁻(H₂O)ₙ+O₂^{-(H}₂O)_{n′}→H₂O₂+O²+(m+m′+n+n′)H₂O (3)

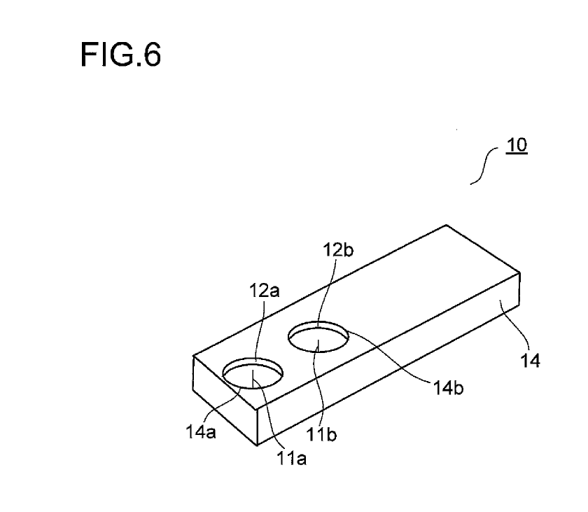

Alternatively, as illustrated in FIG. 6, the ion generating unit 10 may be formed so as to have a flat plate shape and arranged on an inner surface of the casing 2.

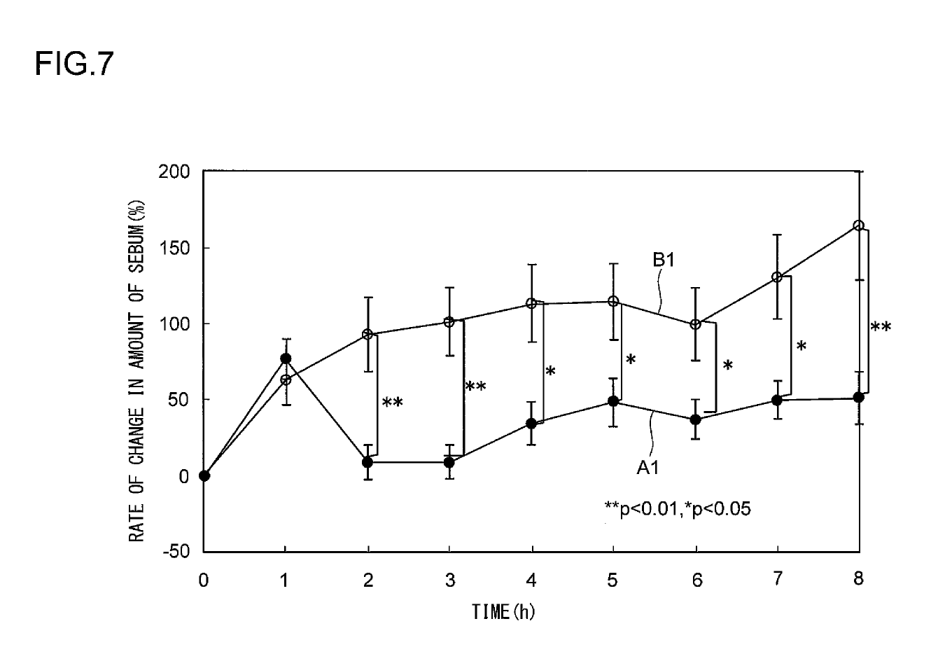

FIG. 7 shows test results when a change in the amount of sebum on the facial skin surface was examined while generating positive ions and negative ions in a room. The vertical axis represents the rate of change (unit: %) in the amount of sebum, and the horizontal axis represents time (unit: h). In this test, 17 subjects stayed in the room. In the figure, A1 shows the results when the ions were generated such that the concentration of the positive ions and the concentration of the negative ions were each 200,000/cm³ in the vicinity of the faces of the subjects. For comparison, the results when no ions were generated are shown by B1 in the figure.

Referring to the figure, in the case where no ions were generated, after eight hours passed from the start of the test, the rate of increase in the amount of sebum was 150% or more. In contrast, in the case where the ions were generated, after eight hours passed from the start of the test, the rate of increase in the amount of sebum was suppressed to about 50%. Thus, an increase in sebum can be suppressed by emitting the positive ions and the negative ions toward the skin surface. Note that, in the figure, symbols [**] and [*] represent the p-value in a statistical test, and respectively represent p<0.01 and p<0.05.

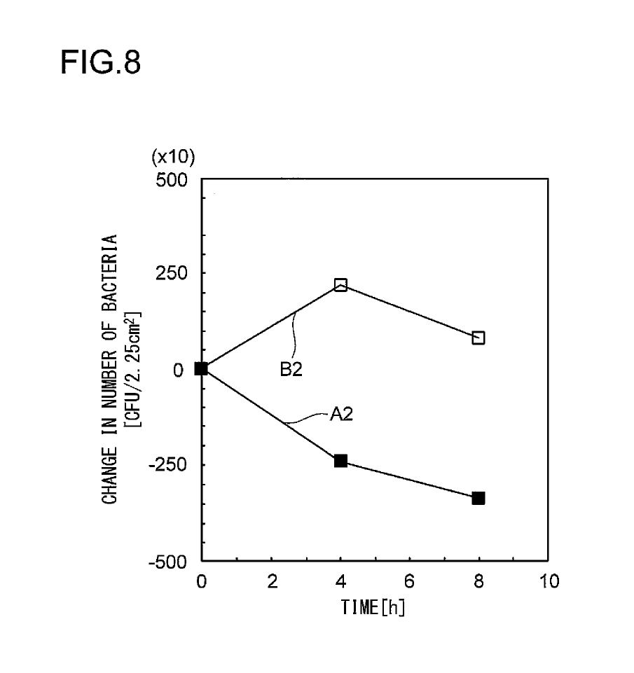

FIG. 8 shows test results when a change in the number of Staphylococcus aureus bacteria on the facial skin surface was examined while generating positive ions and negative ions in a room. The vertical axis represents the change in the number of Staphylococcus aureus bacteria (unit: CFU/2.25 cm²), and the horizontal axis represents time (unit: h). It is believed that Staphylococcus aureus, which is known as a harmful bacterium, is one of causes of skin roughening and relates to skin conditions such as pimples, impetigo, and atopic dermatitis.

In this test, 15 subjects stayed in the room having an area corresponding to 6 tatami-mats (floor area: 9.8 m²) where a temperature of 26° C. to 28° C. and a humidity of 40% to 60% were maintained. In the figure, A2 shows the results when the ions were generated such that the concentration of the positive ions and the concentration of the negative ions were each 100,000/cm³ in the vicinity of the faces of the subjects. For comparison, the results when no ions were generated are shown by B2 in the figure.

Referring to the figure, in the case where no ions were generated, the number of Staphylococcus aureus bacteria increased after 4 hours and 8 hours from the start of the test. In contrast, in the case where the ions were generated, the number of Staphylococcus aureus bacteria decreased after 4 hours and 8 hours from the start of the test. Thus, by emitting the positive ions and the negative ions toward the skin surface, active species generated from the ions can decompose Staphylococcus aureus to suppress the proliferation of Staphylococcus aureus.

In this test, a change in the number of Staphylococcus epidermidis bacteria was also examined. According to the results, there was no difference in the number of bacteria between the case where ions were generated and the case where no ions were generated. It was demonstrated that Staphylococcus epidermidis, which is believed to be a beneficial bacterium that prevents pathogenic bacteria from entering the body, is not affected by the influence of the ions.

The test results shown in FIGS. 7 and 8 show that, by bringing the positive ions and the negative ions into contact with the skin surface, an increase in sebum can be suppressed and Staphylococcus aureus can be decomposed to suppress proliferation thereof. Consequently, pores of the skin are not conspicuous and skin roughening is prevented, thus achieving an effect of skin care. Accordingly, by driving the ion generating unit 10 while closing the open surface 2a of the casing 2 of the skin care device 1 with the skin surface, the positive ions and the negative ions can be easily brought into contact with the skin surface to achieve an effect of skin care.

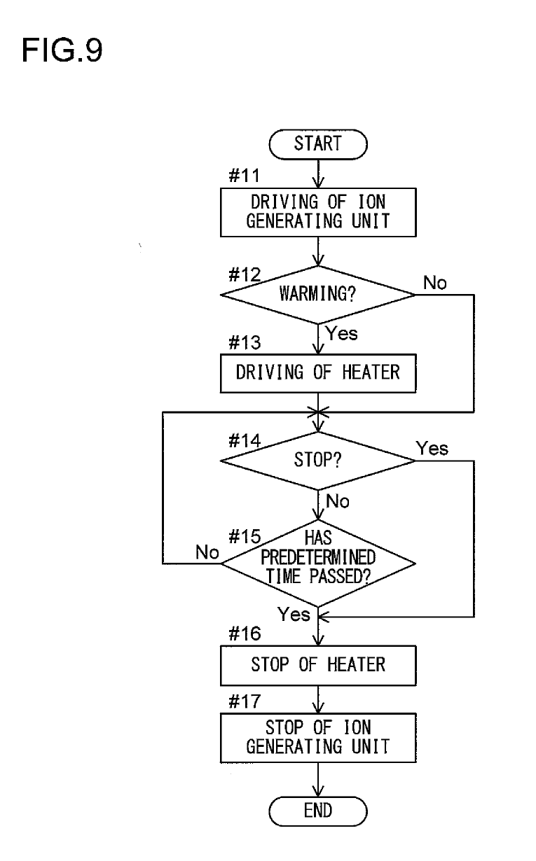

FIG. 9 is a flowchart illustrating an operation of the skin care device 1. When the casing 2 is brought into contact with a skin surface and the operation is started, the ion generating unit 10 is driven in step #11. Consequently, positive ions and negative ions naturally diffuse in the casing 2 and fill the casing 2. The positive ions and the negative ions contact the skin surface, and active species generated by these ions also contact the skin surface. Thus, an increase in sebum on the skin surface is suppressed and Staphylococcus aureus is decomposed.

In step #12, whether or not warming with the heater 6 is set to be performed is determined. In the case where warming is set not to be performed, the process advances to step #14. In the case where warming is set to be performed, the heater 6 is driven in step #13. Accordingly, pores on the skin surface are opened, and an increase in sebum in the pores is suppressed.

In step #14, whether or not an operation of stopping the operation has been performed is determined. In the case where the operation of stopping the operation has not been performed, the process advances to step #15 and whether a predetermined time (for example, 15 minutes) has passed or not is determined. In the case where the predetermined time has not passed, steps #14 and #15 are repeated. In the case where the operation of stopping the operation has been performed or the predetermined time has passed, the process advances to step #16 and the heater 6 is stopped. In step #17, the ion generating unit 10 is stopped to finish the operation of the skin care device 1.

According to the present embodiment, since positive ions and negative ions are generated and brought into contact with a skin surface while the open surface 2a of the casing 2 is closed by the skin surface, the conspicuousness of pores can be suppressed while the stress on the skin is low and an increase in sebum is easily suppressed. Furthermore, active species generated by the ions decompose Staphylococcus aureus on the skin surface, thus preventing skin roughening. Accordingly, an effect of skin care can be easily achieved by pressing the casing 2 on a desired position of the skin surface.

Furthermore, since the positive ions are ions represented by H⁺(H₂O)ₘ and the negative ions are ions represented by 0₂⁻(H₂O)ₙ (where m and n are each any natural number), an increase in sebum on the skin surface can be reliably suppressed and Staphylococcus aureus can be decomposed.

Furthermore, since the ions are allowed to naturally diffuse in the casing 2, blowing with a fan or the like is not conducted and thus drying of the skin surface can be prevented.

Furthermore, since the heater 6 (warming device) that warms the skin surface, which contacts ions, is provided, pores are opened to suppress an increase in sebum in the pores.

Furthermore, since the casing has a cup shape and a peripheral edge of the open surface is pressed onto the skin surface by holding the casing, an effect of skin care can be achieved by easily bringing ions into contact with the skin surface.

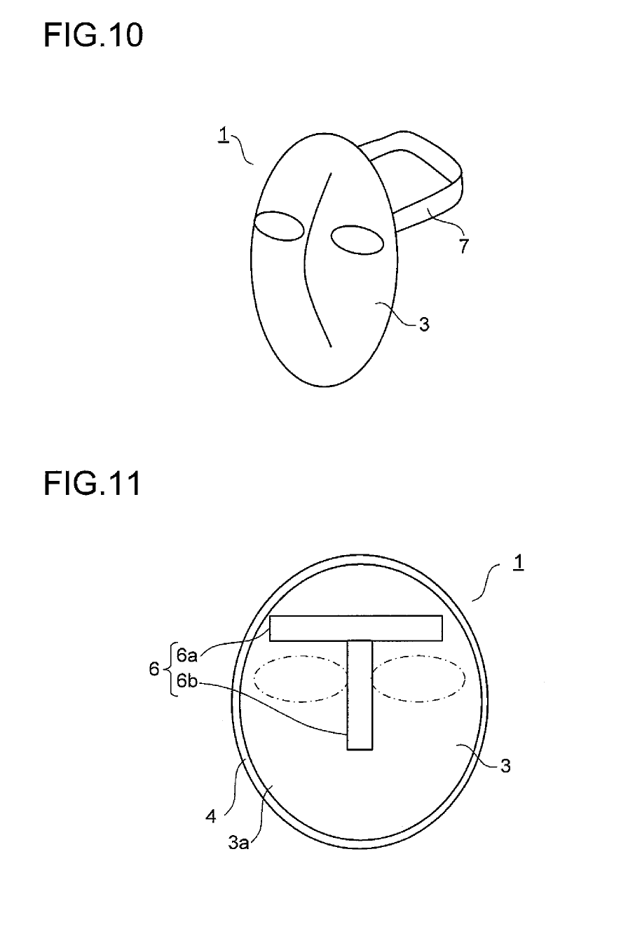

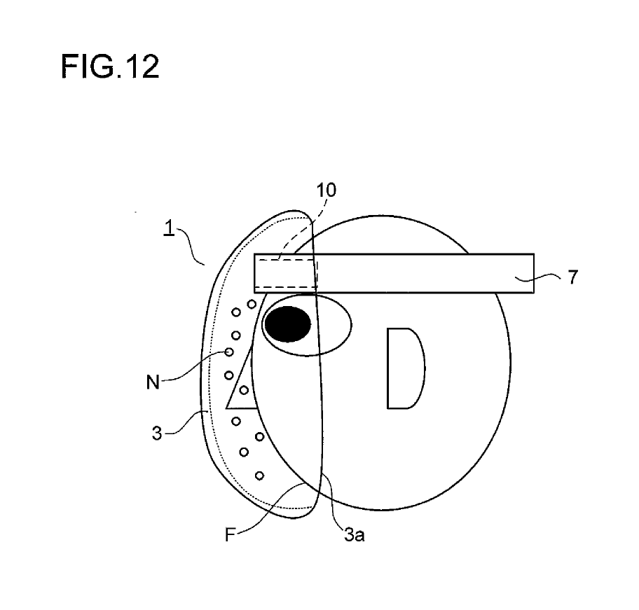

Next, FIGS. 10 and 11 are respectively a perspective view and a back view of a skin care device 1 according to a second embodiment. For the sake of convenience of explanation, parts the same as those in the first embodiment illustrated in FIGS. 1 to 8 are assigned the same reference numerals. The skin care device 1 includes a mask-like casing 3 that covers a human face F (refer to FIG. 12). A belt 7 that is fitted to the head of a user to hold the skin care device 1 in place is provided on an upper portion of the casing 3. The back surface of the casing 3 forms an open surface 3a, and packing 4 composed of an elastomer such as silicone rubber is provided around the periphery of the open surface 3a.

A heater 6 is arranged on the inner surface of the casing 3. The heater 6 is arranged so as to have a T-letter shape having a forehead portion 6a facing the forehead of the user and extending in the horizontal direction and a nose portion 6b facing the nose and extending in the vertical direction. Driving of the heater 6 opens pores over the entire face, mainly in a forehead portion and a nose portion where the amount of secretion of sebum is large. The heater 6 is formed by, for example, being applied onto the inner surface of the casing 3 or being embedded in the casing 3. The heater 6 may be a film-shaped heating element, a line-shaped heating element covered with a silicone rubber film, or the like.

FIG. 12 is a side view illustrating a state in which the skin care device 1 is worn. When the belt 7 is fitted to the head so that the packing 4 closely contacts the periphery of the face F, the open surface 3a is closed to seal the inside of the casing 3. In this case, a certain space is formed between the inner surface of the casing 3 and the face F.

An ion generating unit 10 that is similar to that of the first embodiment is arranged on a front portion of the belt 7 and generates positive ions and negative ions. Thus, ions N naturally diffuse in the casing 3 and fill the casing 3. The positive ions and the negative ions contact the skin surface, and active species generated by the ions also contact the skin surface. Thus, an increase in sebum is suppressed and Staphylococcus aureus is decomposed.

According to the present embodiment, as in the first embodiment, since positive ions and negative ions are generated and brought into contact with a skin surface while the open surface 3a of the casing 3 is closed by the skin surface, the conspicuousness of pores can be suppressed while the stress on the skin is low and an increase in sebum is easily suppressed. Furthermore, active species generated by the ions decompose Staphylococcus aureus on the skin surface, thus preventing skin roughening. Accordingly, an effect of skin care can be easily achieved by wearing the casing 3 on the face F.

Furthermore, since the ions are allowed to naturally diffuse in the casing 3, blowing with a fan or the like is not conducted and thus drying of the skin surface and the eyes can be prevented.

Furthermore, since the casing 3 has a shape of a mask that covers a human face and the packing 4 is provided around a peripheral edge of the open surface 3a, the inside of the casing 3 is easily sealed and ions can be brought into contact with the entire face.

Furthermore, since the heater 6 is arranged along a forehead portion and a nose portion of the face F, pores in areas where the amount of secretion of sebum is large are opened, thereby further improving the effect of skin care.

### INDUSTRIAL APPLICABILITY

The present invention can be applied to a skin care device for suppressing an increase in sebum of the skin.

### REFERENCE SIGNS LIST

1 skin care device

2,3 casing

2a, 3a open surface

4 packing

6 heater

7 belt

10 ion generating unit

11a, 11b discharge electrode

12a, 12b counter electrode

13 high-voltage power supply

14 body case

1. A skin care method using a closed-bottomed casing having an open surface and an ion generating unit in which discharge is generated by applying a high voltage to generate positive ions and negative ions in the casing, the method comprising driving the ion generating unit while the open surface is closed by a skin surface to seal the inside of the casing; and bringing ions generated in the ion generating unit into contact with the skin surface.

2. The skin care method according to claim 1, wherein the positive ions are represented by H⁺(H₂O)ₘ (where m is any natural number) and the negative ions are represented by O₂⁻(H₂O)ₙ (where n is any natural number).

3. The skin care method according to claim 1, wherein the ions are allowed to naturally diffuse in the casing.

4. The skin care method according to claim 1, wherein the ions are generated while the skin surface is warmed.

5. A skin care device comprising a closed-bottomed casing having an open surface; and an ion generating unit in which discharge is generated by applying a high voltage to generate positive ions and negative ions in the casing, wherein ions are generated from the ion generating unit which is driven while the inside of the casing is sealed by closing the open surface with a skin surface to bring the ions into contact with the skin surface.

6. The skin care device according to claim 5, wherein the positive ions are represented by H⁺(H₂O)ₘ (where m is any natural number) and the negative ions are represented by O₂⁻(H₂O)ₙ (where n is any natural number).

7. The skin care device according to claim 5, wherein the ions are allowed to naturally diffuse in the casing.

8. The skin care device according to claim 5, further comprising a warming device that warms the skin surface that contacts the ions.

9. The skin care device according to claim 8, wherein the casing has a shape of a mask that covers a human face, and the warming device is arranged along a forehead portion and a nose portion of the face.

10. The skin care device according to claim 5, wherein the casing has a cup shape, and a peripheral edge of the open surface is pressed onto the skin surface by holding the casing.

11. The skin care device according to claim 5, wherein the casing has a shape of a mask that covers a human face, and packing is provided around a peripheral edge of the open surface.

12. The skin care method according to claim 2, wherein the ions are allowed to naturally diffuse in the casing.

13. The skin care method according to claim 2, wherein the ions are generated while the skin surface is warmed.

14. The skin care device according to claim 6, wherein the ions are allowed to naturally diffuse in the casing.

15. The skin care device according to claim 6, further comprising a warming device that warms the skin surface that contacts the ions.

16. The skin care device according to claim 6, wherein the casing has a cup shape, and a peripheral edge of the open surface is pressed onto the skin surface by holding the casing.

17. The skin care device according to claim 6, wherein the casing has a shape of a mask that covers a human face, and packing is provided around a peripheral edge of the open surface.
